(19) United States
(12) Patent Application Publication
(10) Pub. No.: US 2019/0392952 A1
(43) Pub. Date: Dec. 26, 2019

(54) COMPUTER-IMPLEMENTED METHODS, SYSTEMS, AND COMPUTER-READABLE MEDIA FOR DIAGNOSING A CONDITION

(71) Applicant: Basil Leaf Technologies, LLC, Paoli, PA (US)

(72) Inventors: Basil M. Harris, Paoli, PA (US); George C. Harris, Ramsey, NJ (US); Philip J. Charron, Philadelphia, PA (US); Julia D. Harris, Hermitage, TN (US); Andrew D. Singer, Hopkinton, MA (US); Constantine F. Harris, Wyomissing, PA (US); Edward L. Hepler, Malvern, PA (US)

(73) Assignee: Basil Leaf Technologies, LLC, Paoli, PA (US)

(21) Appl. No.: 16/465,327

(22) PCT Filed: Nov. 28, 2017
(86) PCT No.: PCT/US17/63494

§ 371 Date: May 30, 2019

Related U.S. Application Data
(60) Provisional application No. 62/432,188, filed on Dec. 9, 2016.

(51) Int. Cl. G16H 50/20; G16H 15/00; G16H 10/60

(52) U.S. Cl. CPC G16H 50/20; G16H 10/60; G16H 15/00

(57) ABSTRACT

One aspect of the invention provides a computer-implemented method for diagnosing a condition. The computer-implemented method includes: (a) receiving one or more inputs regarding a subject's symptoms; (b) updating a plurality of models in parallel based on the one or more inputs, each model generating a numerical score reflecting a likelihood of one of a plurality of conditions; (c) identifying one or more most-likely conditions as a function of the numerical scores produced by the models; (d) requesting additional input based on the most-likely conditions; (e) receiving the additional input; (f) updating the models in parallel based on the additional input; (g) comparing updated numerical scores or a difference between sequenced updated numerical scores to a stored confidence threshold; and (h) repeating steps (c)-(g) until the compared numerical scores or the difference between sequenced numerical scores exceeds the stored confidence threshold.

### CROSS-REFERENCE TO RELATED APPLICATION

This application claims the benefit of priority of U.S. Provisional Patent Application No. 62/432,188, filed Dec. 9, 2016, the entire disclosure of which is hereby incorporated herein by reference.

### FIELD OF THE INVENTION

The present invention relates generally to computerized medical diagnostic systems, and more particularly, to a computerized system for diagnosing a condition using knowledge-based scoring.

### BACKGROUND OF THE INVENTION

Medical care remains both resource-intensive and resource-constrained. Patients often face lengthy waits to see a medical professional and medical professionals are under tremendous time constraints when interacting with patients. Additionally, many patients face geographic and/or temporal barriers to visiting a medical professional.

### SUMMARY

One aspect of the invention provides a computer-implemented method for diagnosing a condition on a computer including a processor, memory, at least one of a user interface and a communication interface, a user profile module, a question module, a vitals module, and a diagnostic module. The computer-implemented method includes: (a) controlling the processor and one or more of the user interface, the communication interface, the user profile module, the question module, and the vitals module on the computer to receive one or more inputs regarding a subject's symptoms; (b) controlling the processor and the diagnostic module on the computer to update a plurality of models stored in the memory on the computer in parallel based on the one or more inputs, each model generating a numerical score reflecting a likelihood of one of a plurality of conditions; (c) controlling the processor and the diagnostic module on the computer to identify one or more most-likely conditions as a function of the numerical scores produced by the models stored in the memory on the computer; (d) controlling the processor, the question module, and at least of the user interface and the communication interface on the computer to request additional input based on the most-likely conditions; (e) controlling the processor, the question module, and at least of the user interface and the communication interface on the computer to receive the additional input; (f) controlling the processor and the diagnostic module on the computer to update the models stored in the memory on the computer in parallel based on the additional input; (g) controlling the processor and the diagnostic module on the computer to compare updated numerical scores or a difference between sequenced updated numerical scores to a stored confidence threshold; and (h) controlling the processor and the diagnostic module and the question module on the computer to repeat steps (c)-(g) until the compared numerical scores or the difference between sequenced numerical scores exceeds the stored confidence threshold.

The models can be weighted summations of the inputs. The weighted summations can include negative, zero, and positive weightings.

The models can produce a numerical score. The confidence threshold can be a predefined number that the numerical score must equal or exceed. The confidence threshold can be a predefined difference between the numerical scores of a most-likely condition and a next-most-likely condition.

The computer-implemented method can further include: (h) receiving one or more external inputs; and (i) updating the models based on the one or more external inputs. The one or more external inputs can include one or more selected from the group consisting of: epidemiological data, subject-entered data, and electronically obtained data. The electronically obtained data can be periodically updated.

Another aspect of the invention provides a computer-implemented method for diagnosing a condition on a computer including a processor, memory, at least one of a user interface and a communication interface, a user profile module, a question module, a vitals module, and a diagnostic module. The computer-implemented method includes: (a) controlling the processor and one or more of the user interface, the communication interface, the user profile module, the question module, and the vitals module on the computer to obtain one or more subject inputs regarding a subject; (b) controlling the processor and one or more of the user interface, the communication interface, the user profile module, the question module, and the vitals module on the computer to obtain one or more family inputs regarding the subject's family; (c) controlling the processor and one or more of the user interface, the communication interface, the user profile module, the question module, and the vitals module on the computer to obtain one or more symptom inputs regarding the subject's symptoms; (d) controlling the processor and the diagnostic module on the computer to update models stored in the memory on the computer for a plurality of conditions based on the one or more subject inputs, family inputs, and symptom inputs; (e) controlling the processor and the diagnostic module on the computer to identify m most-likely diagnoses based on the models stored in the memory on the computer; (f) controlling the processor and at least one of the diagnostic module and the question module on the computer to identify n most-influential questions for the m most-likely diagnoses based on previously assigned weights associated between the m most-likely diagnoses and a plurality of questions; (g) controlling the processor, the question module, and at least of the user interface and the communication interface to present the n most-influential questions to the subject; (h) controlling the processor, the question module, and at least of the user interface and the communication interface on the computer to obtain responses to at least one of the n most-influential questions; (i) controlling the processor and the diagnostic module on the computer to update the models stored in the memory on the computer based on the obtained responses; (j) controlling the processor and the diagnostic module on the computer to update the m most-likely diagnoses based on the models stored in the memory on the computer; (k) controlling the processor and the diagnostic module on the computer to determine whether a most-likely diagnosis exceeds a confidence threshold and: (1) if so, controlling the processor and at least of the user interface and the communication interface on the computer to present the most-likely diagnosis to the subject; and (2) if not, controlling the processor and the diagnostic module and the question module on the computer to repeat steps (f)-(k).

Another aspect of the invention provides a system for diagnosing a condition. The system includes: a user profile module; a diagnostic module; and a question module. The user profile module includes computer-readable program code including steps for: obtaining one or more user profile inputs regarding a subject's medical status and history; and recording the one or more user profile inputs. The vitals module comprising computer-readable program code including steps for: receiving one or more vitals inputs from one or more sources selected from the group consisting of: sensors and laboratories; determining whether the one or more vitals inputs are clinically significant; and if the one or more inputs are clinically significant, recording the one or more vitals inputs. The diagnostic module includes computer-readable program code including steps for: populating and updating a plurality of diagnosis modules based on the one or more user profile inputs stored by the user profile module and the one or more vitals inputs stored by the vitals module; identifying m most-likely diagnoses based on the diagnosis models; updating the plurality of diagnosis models based on responses to questions posed to the subject by the question module and any further vitals inputs; updating the m most-likely diagnoses based on the models; determining whether a most-likely diagnosis exceeds a confidence threshold and: if so, presenting the most-likely diagnosis to the subject; and if not, instructing the question module to ask further questions based on the updated m most-likely diagnoses. The question module comprising computer-readable program code including steps for: identifying n most-influential questions for the m most-likely diagnoses based on previously assigned weights associated between the m most-likely diagnoses and a plurality of questions; controlling a user interface to present the n most-influential questions to the subject; obtaining responses to at least one of the n most-influential questions; and recording the responses.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIGS. 1A and 1B depict a diagnostic system according to an embodiment of the invention.

FIGS. 2 and 3 depict diagnostic methods according to embodiments of the invention.

FIG. 4 depicts a sample medical report provided to a user according to an embodiment of the invention.

FIGS. 5A and 5B depict a sample medical report provided to a medical professional according to an embodiment of the invention.

FIG. 6 depicts a user using an auscultation device in communication with a tablet computer running a diagnostic method according to an embodiment of the invention.

FIG. 7 depicts interactions between a diagnostic method/system (referred to under the DXTER™ trademark) and a user, sensors, and internet resources according to an embodiment of the invention.

FIG. 8 depicts interactions between a diagnostic method/system (referred to under the DXTER™ trademark) and a user and other actors according to an embodiment of the invention.

FIGS. 9 and 10 depict diagnostic methods according to embodiments of the invention.

FIGS. 11A-11C depict personas used by physicians during testing of an embodiment of the invention in Working Example 2.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

As used herein, the term “condition” includes diseases, abnormal conditions, or disorders of a structure or function that affects part or all of an organism.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### Detailed Description

Embodiments of the invention provide a medical diagnostic system incorporating unique examination and diagnostic algorithms and synthesizing data, requesting further inputs, making a diagnosis, and suggesting a course of action. Data collection, processing, and integration into unique diagnostic algorithms can all occur locally on the system.

The process can begin with the user's focused medical problem, i.e., the chief complaint. The artificial intelligence of the medical diagnostic module can guide the user through a unique path of queries and physical interactions depending upon the user's responses and the gathered data. When interacting with the system, there can be multiple potential paths for the data to be collected. As more information is collected, the diagnostic module can narrow the field of the most-likely diagnoses. The ranking of the differential diagnoses can be influenced by all of the subjective and objective inputs. This dynamic process can further guide the path of requested data until enough information is acquired to provide a diagnosis or sufficiently exclude other diagnoses.

Over the course of a session, interaction with the device can flow much like a physician visit or a series of visits, incorporating a brief series of queries, physical exam elements, vital signs sensor data acquisition, and, when needed, more advanced point-of-care analyses. Navigation through the system is unique for each session and can be a function of the many inputs, with each session's unique path decided dynamically by the medical diagnostic systems and methods described herein. The user can be kept engaged and abreast of the process as data are gathered and analyzed.

### Deconstructing and Reconstructing the Diagnostic Process

Drawing on years of experience in clinical emergency medicine, Applicant created a workflow based on a set of chief complaints (e.g., headache, abdominal pain, cough, etc.). Applicant formulated a specific set of questions, a flow of queries, and critical data needed to resolve each of the identified complaints. Additionally, Applicant formulated a system for assigning metric values to each diagnosis as data is collected. These flowcharts were then merged to create a master list of questions and data that would be needed to accurately determine a diagnosis.

Each step within the workflow was assigned branching logic and a metric value for each of the diagnoses. After looking at the metrics holistically, each of the diagnoses' metric values was normalized, so that diagnosis values would be easily compared with each other. As needed, metrics were added, removed, or combined to provide a higher likelihood in separating one diagnosis from another. The diagnosis can be further informed by accessing local and regional health data (e.g., current prevalence of certain diseases such as influenza, pertussis, zika, sexually transmitted diseases, Middle East Respiratory Syndrome, coronavirus, ebola, and other exotic outbreaks obtained from health authorities such as the Centers for Disease Control and Prevention, local weather conditions, pollen counts that may affect respiratory ailments, and the like). This information, when taken into consideration along with all of the subjective and objective data obtained, can impact the likelihood of certain diagnoses.

The subjective data obtained from the focused queries can be similar to that of a typical physician and/or Emergency Department (ED) visit and follow the same flow. As data are collected, the list of presented questions will expand or contract, depending on the user's responses and acquired sensor data. For example, an objective finding of a fever may trigger a line of inquiry regardless of whether or not the user indicated it as a problem.

The basic objective data can be obtained from unique routines of the physical examination and vital signs. Embodiments of the invention can employ many novel techniques to acquire objective information via the interaction between the user and a computing device (e.g., a tablet computer). For example, a neurological examination can be performed (when indicated by the clinical scenario) that leverages speech recognition algorithms, digital photography, and a touch screen to assess the presence of neurological deficits. Applicant believes that this system may provide a more systematic and detailed stroke assessment than is typically provided by many physicians.

The system is able to accept data from many inputs. Most of the sub-system components connect and transfer data via a wireless (e.g., BLUETOOTH®) connection. The peripheral components can include specific devices for measurement of vital signs and for acquiring further elements of the physical examination. Additional point-of-care devices can be incorporated into the systems and methods described herein in order to obtain specific information, such as urinalysis or blood analysis.

### System Overview

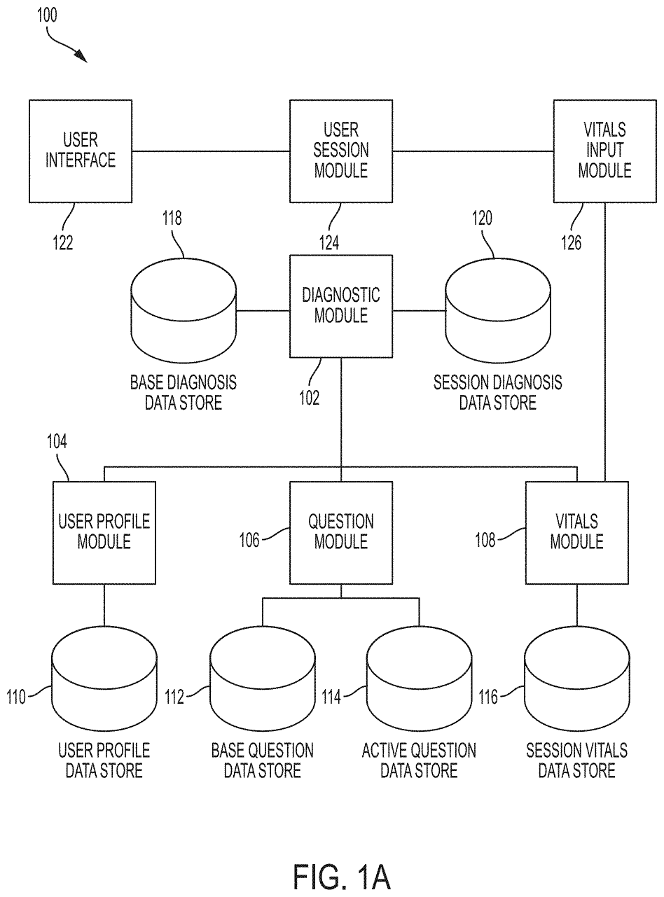

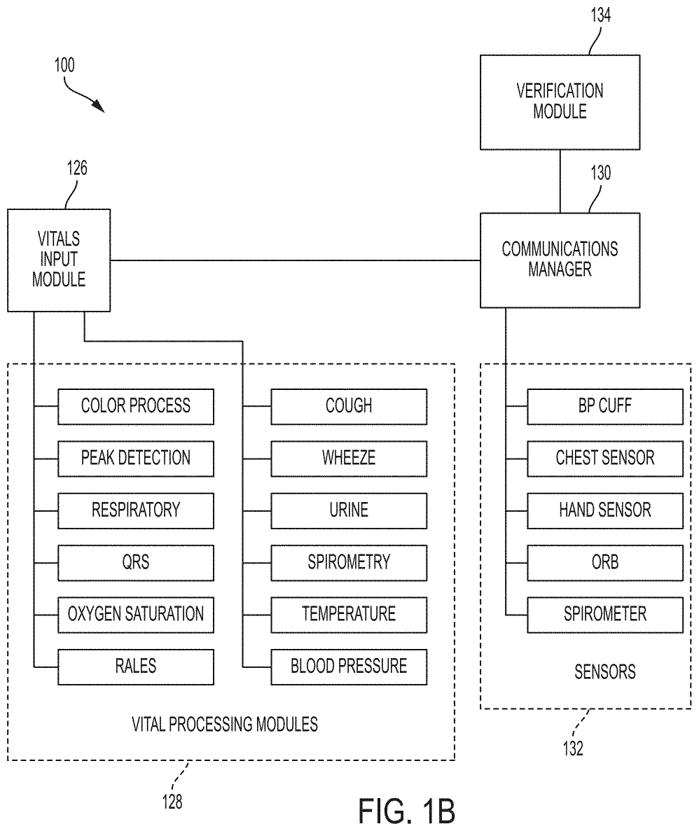

Referring to FIGS. 1A and 1B, embodiments of the invention provide a system 100 for diagnosing a condition. Embodiments of the invention can be designed and implemented in modular or component-based architecture in which individual modules have well-defined functions and interfaces. Although described in this manner, embodiments of the invention can also be implemented using other software and/or hardware design principles and techniques.

The modules 102, 104, 106, 108, 122, 124, 126, 128, 130, 134 described and depicted herein can be implemented as separate processes and/or threads on one or more physical devices (e.g., computers, servers, tablets, smartphones, and the like). Likewise, one or more modules can be implemented on one or more physically remote computers. For example, user interface module 122, user session module 124, and vitals input module 126 can be implemented on a device (e.g., a smartphone or tablet computer) local to the user, while other components (e.g., diagnostic module 102) can be implemented on a remote server based on inputs received from the user's device.

A diagnostic module 102 can be the central hub that correlates the interpreted physical data being sent into the vitals module 108's data store 116, the active question data store 114, and the active user profile data store 110.

The diagnostic module 102 according to an embodiment of the invention can utilize user profile module 104, question module 106, and vitals module 108 as input sources in order to achieve a diagnosis. Each of these sub-modules 104, 106, 108 is responsible for managing different user data elements and presenting their information to the diagnostic module 102 for session analysis.

Each of the sub-modules 104, 106, 108 can utilize a slightly different model in dealing with their internal data structures. These differences between how these elements can be processed can be based on each data set's dynamics.

### User Profile Data Structures

User profile data does not change frequently and can be made up of the user's medical history (e.g., both the user's and her family's). The user interacts with this data structure during the configuration of her profile. This data can be essentially static when a session starts. For example, the user's weight is unlikely to change during the session.

Almost all of the user profile data elements can be stored as numbers and represent binary Yes/No values to simple medical history questions (0=No and 1=Yes), for example, “Does your family have a history of heart disease?”. Date of birth, height, weight, and body mass index are exceptions to this format.

Exemplary user profile data structures are provided in Appendix A, in which NSNumber, NSDate, and NSString are classes in the Objective-C programming language. Each user can be assigned a unique identifier and then allowed to fill in the user profile. In some embodiments, date of birth, sex, height and weight are needed before a session can move forward.

### Active Session Question Response Data Structures

Active session question response data can be (almost solely) based on the user's responses to questions revolving around the current diagnosis state and the user's chief complaint(s) selection at the start of the session. As the user responds, different questions can be presented and, based on those responses, follow-up questions can be submitted. Additionally, questions can be generated based on the user's vitals that are being monitored. Deviation from a normal base-line vital reading can trigger follow-up questions. For example, if the user develops a fever during the session, the diagnostic module 102 and/or question module 106 can select target questions regarding the user's newly discovered fever.

As depicted in Appendix B, Question Module data can be stored as an immutable array of dictionaries. Each unique data element can be an immutable key/value combination that describes a particular question or test to which the user should respond. This data structure can be copied into the user's active question data store 114 as a mutable data structure. This data structure is now amendable based upon user's responses to the presented questions.

A question data structure can be empty at the start of a session and can be considered static after a user's response, but users can be given the opportunity to alter their responses. Each data element's response can be stored as a string of text.

The majority of the question module's data elements can be single-choice options. These elements can be stored as a string version of the selected option (e.g., “0001” or “0003”).

Several questions allow for multiple selections. For example, a “comma” can separate these selections (e.g., “0002, 0004, 0006”).

Additionally, some questions can require a more complex response to be analyzed such as dealing with visual, audio or aural problems. These questions can store the user's results as a string interpretation of one or more floating-point numbers (e.g., “12.45” or “34.12, 184.5”).

### Active Session Vitals and Lab Test Results Data Structure

Active session vitals and lab test result data can be highly dynamic. As the various sensors 132 that monitor the user's vitals record events, the data can be added to the session vitals data store 116 by the vitals module 108. The user need not directly interact with the vitals module 108, but the sensors 132 that are monitoring the user can. One embodiment of the invention currently tracks 74 unique vital elements. Each element can have multiple vital events.

As illustrated in Appendix C, vitals module 108 can be stored in a mutable array of dictionaries. Each unique data element can be an array of key/value dictionary entries. As results are recorded to a specific data element, a new, immutable, dictionary element can be added that reflects the recorded values.

The recorded vital event can contain a timestamp, a monitoring device tag ID (e.g., device name, serial number and software version running on the device), and the recorded event value. Vital events can be integers, floats, or a single selection from a menu of choices. Each of the Vital Module's data elements can allow for data to be entered multiple times during the running session. For example, the Heart Rate Variability (HRV) data element can have 5 choices: No Value, Low, Moderate, High or No Variation. As the physical hardware is monitoring the user through the vitals input module 126 that acts as bridge between the physical hardware and the vitals module 108, data can be inserted into the vital module's HRV data element. The vitals input module 126 can decide if the data being seen on the hardware meets the required criteria and then record a vitals module entry with the appropriate value, timestamp and monitoring device's ID information. For example, the vitals module 108 can determine whether the data is clinically significant (e.g., likely to impact a diagnosis). Clinical significance can be assessed based on boundaries as discussed below and can also be based on variance from prior data. For example, although a 102.1° F. temperature would impact many diagnoses, such data may not be deemed clinically significant if a 102.2° F. temperature was recorded 10 minutes prior.

Each vitals module data element can be tagged with several boundary options to ensure data validity and to help the diagnostic module 102 draw appropriate conclusions. Elements can also have minimum and/or maximum boundaries to prevent unrealistic values from entering into the calculations. Data points can also be flagged as a maximum, minimum, or average value. These tags allow for the diagnosis module to only look at a specific data element's data points' minimum, maximum or average when incorporating that element into a diagnosis. An example of this would be Vital Number 7: Blood Pressure Systolic Maximum. Every BP reading can be recorded, but this particular data element is the running maximum value across all BP readings that were taken during the session. Other exemplary vital signs can include temperature, heart rate, respiratory rate, oxygen saturation, and the like.

### Diagnostic Data Structure

Diagnostic module results can be based on the three data structure inputs being observed and computed through each of the module's active diagnoses. The resulting tabulations can then be compared to assign a diagnosis ranking, along with distance vectors between diagnoses being used to further separate the active diagnosis selection(s) from the inactive ones.

The diagnostic module data structure can be a mutable array of key/value dictionaries that define each diagnosis based on the three data inputs. The diagnosesVitalMatrix and the diagnosesQuestionMatrix dictionaries can hold the description of a given condition, based on point values being assigned to a user's medical history, her answers to questions, and what the device hardware is reading for their current vitals.

An exemplary sample diagnostics data structure is provided in Appendix D.

As the three data input modules collect data, the diagnosis module can constantly update the running score for each diagnosis. These values can be used to determine the next line of questions and assess if the user has met enough of the criteria to be diagnosed for any particular disease. For example, a particular datum (e.g., blood glucose level, blood type) can affect the probability of n diagnoses to varying degrees and can be stored as a vector of probability influence weights [1.0, −0.5, 0, . . . , pₙ] that can be added to update a plurality of n diagnoses. The actual weights can vary between implementations. The probability influence weights can be stored in one or more databases and/or data structures, e.g., in a rule/action or IF/THEN format. For example, base diagnosis data store 118 can store a plurality of rules instructing the diagnostic module 102 to modify one or more probabilities based on a detected condition. The running probabilities for a particular patient can be stored in the session diagnosis data store 120.

### Diagnostic Methods

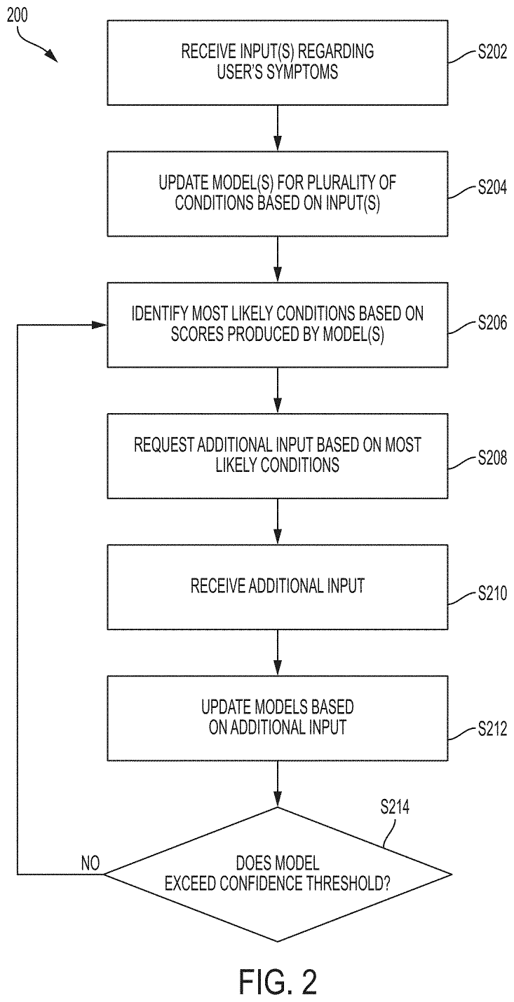

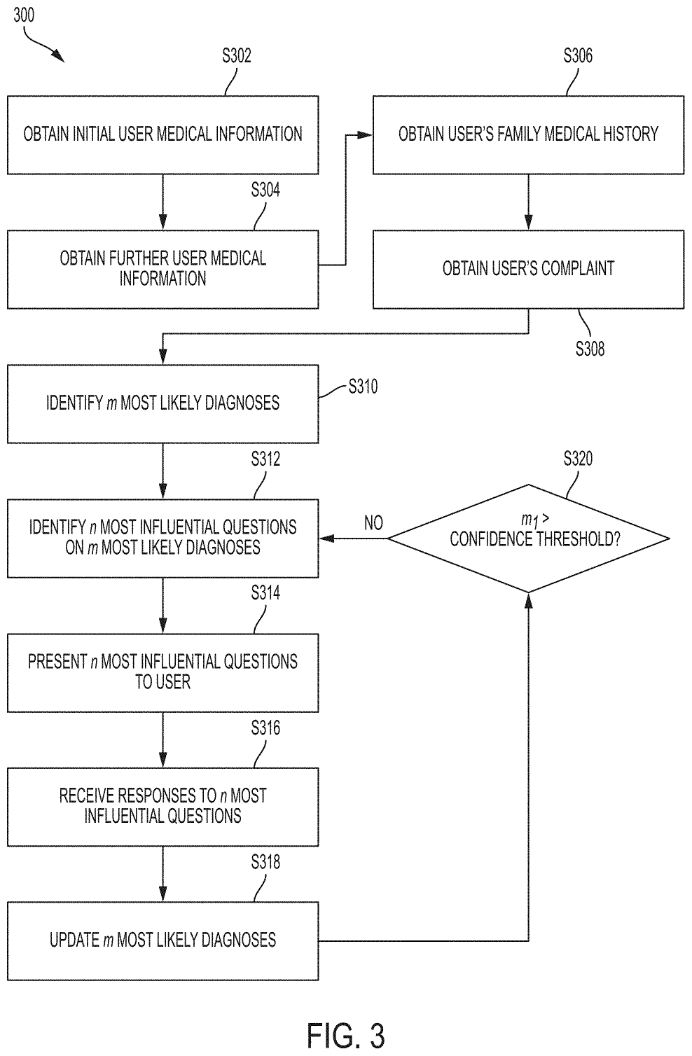

Referring now to FIGS. 2, 3, and 9, embodiments of the invention provide methods 200, 300, 900 of diagnosing a condition.

Diagnostic methods 200, 300 can be seen as 6 phases with a 7th input constantly or periodically received from the vitals input module 126. These phases can be: Initial User Information, User's Personal Medical History, User's Family Medical History, Chief Complaint Selection, Chief Complaint Question Module, and Review of Secondary Questions. While the user progresses through each of these steps, telemetry and lab information can also being fed into the diagnosis module 102 for analysis.

During every phase's individual sub-steps, the diagnostic module 102 can review all collected data and attempt to reach a diagnosis. Absent a clear winner, the diagnosis module can return an absence of condition result. Even if the diagnostic module 102 does not have a clear diagnosis winner, it can use the current, leading diagnoses to help guide the question module's next line of inquiries.

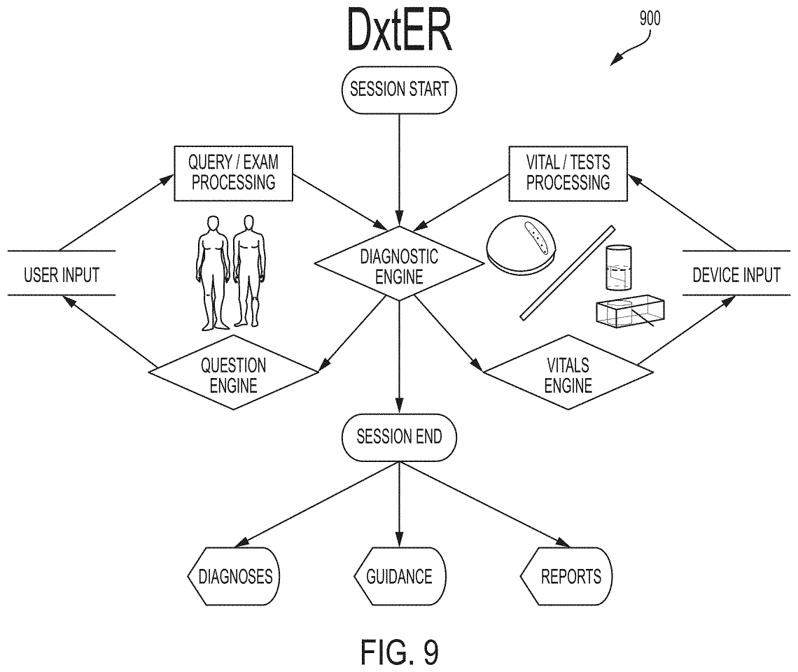

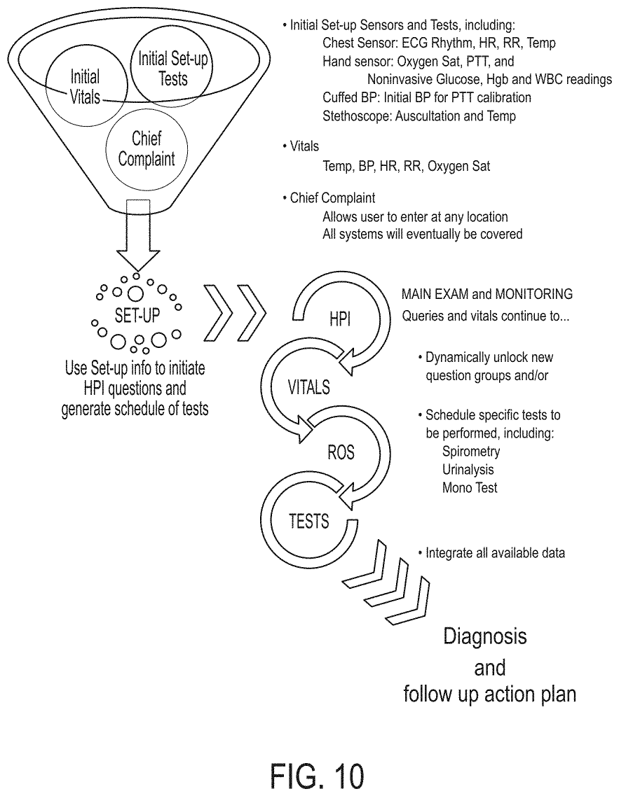

At the initial session start depicted in FIG. 9, when all diagnoses values are set to “zero”, the question module 106 can obtain data in the following order: (1) basic user information (fixed order), (2) user's personal medical history (fixed order), (3) user's family medical history (fixed order), (4) user's complaint(s)—the current reason that they are using the system (fixed order), and (5a and 5b) user's current state of health (variable order).

Absent any diagnosis leader, the question module 102 can focus the line of questions into the following order: fever, cough, ear, throat, breathing, urinary, abdominal, fatigue, skin, chest, heart, speech, weakness, arm, leg, eyes, headache, disorientation, neck, back, and genitals.

User vital signs and labs can be used to alter the running diagnoses, which will, in turn, alter the question module's order of inquiry.

Even though the diagnosis module 102 can tabulate results at every input and regardless of the user's vital sign results, the first 4 phases of data can, in some embodiments, always be asked in the same order for each user session.

### Phase 1—Initial User Information

In Phase 1 (S302), one or more data points regard their medical history can be obtained. Exemplary data points include gender, age, height, weight, body mass index (BMI), and the like.

These data points can be obtained through queries to the user (e.g., through a user interface as discussed herein). In other embodiments, the data points can be obtained from one or more sources such as the user's electronic medical record, a computer application (e.g., APPLE® HEALTHKIT™ software), and the like.

Once any of these data points are entered, a user's diagnosis can immediately start to take shape. For example, age affects several diagnoses, such as chronic obstructive pulmonary disease (COPD) and atrial fibrillation. Also, there are diagnoses that are more prevalent to different genders. Furthermore, height, weight and BMI are all contributing factors to many different diseases, such as diabetes or hypertension.

### Phase 2—User Personal Medical History

After the user's initial user information is obtained, the system can prompt the user to enter additional medical history (S304). Exemplary medical history data points can be binary answers to questions regarding: alcohol use, cocaine use, congestive heart failure, COPD, diabetes, drug use, prior heart attack, heart disease, high blood pressure, HIV, immunosuppressant use, kidney infection, kidney slowing, kidney stones, marijuana use, prescription medication use, rheumatoid arthritis medication use, silicosis, tobacco use, and the like.

Each of these data points affects many different diagnoses and as each data point is provided, the diagnosis module 102 can recalculate the values for the entire range of diagnoses.

### Phase 3—User's Family Medical History

After the user's personal medical history is obtained, the question module 106 can prompt the user to enter family medical history (S306). Exemplary family medical history data points can be binary answers to questions regarding family history of: aneurysm, blood clotting, diabetes, heart attack before age 60, heart disease before age 60, high blood pressure, hypertension, stroke before age 60, sudden death, and the like.

### Phase 4—User's Complaint

In Phase 4, the user can select an area on which the diagnosis should focus (S202, S308). This is the proverbial, “What seems to be the problem?” question. The user's selection(s) can be used to influence an initial line of questions. For example, if a user is complaining about their throat, then the question module 106 preferably will not begin with questions about their arms or legs.

Phase 5a—User Session Question Module Inquiries

Now that the diagnosis module 102 has collected the previously mentioned data points (S202, S204, S302, S304, S306, S308) and the user's vital signs have been streaming into the vitals engine 108, the diagnostic module 102 will have a starting point for a diagnosis (S206, S310). The diagnostic module 102 has for every diagnosis a list of questions and their point values that affect the diagnosis' total score. Some of these are negative, some are positive. The question module 106 is asked to present to the user the next question needed that affects the current top m (e.g., 3) diagnoses (S208, S312, S314). The goal is to have all or a subset of the pertinent questions for the top m diagnoses answered first. These are the most-likely candidates and are also most closely related to the user's stated complaint(s).

Once a question or group of questions is answered (S210, S316), the system can recalculate the point scores for all of the diagnoses (S212), reassign a new “top m” (S206, S318), and then request the next question(s) needed for a full accounting of all of the needed inquiries (S208, S312, S314).

Phase 5b—User Session Question Module Follow-Up Inquires

After the Question Module has exhausted all of the top-m diagnoses' questions, the system can move into follow-up mode. This is also called a final review of systems. It is quite possible (and highly probable) that during Phase 5a, not every focus category's questions will be asked, or even initially addressed. At this point, the question module 106 can present questions from these categories to see if there are any additional data elements that may have been missed that would contribute to a diagnosis that was not in the top m during Phase 5a.

Many of these questions have post requisites and can be used to narrow down the user's issue. The current prototype of the invention includes 284 possible questions that a user could be asked, but it is improbable that they would be exposed to half of them during any given session. Minimally, the user could answer in the negative to all of the presented questions and only be present with about 30 inquires if the patient vital signs do not trigger additional questions.

### Scaling of Diagnosis Algorithm

Mathematically, the worst-case scenario for complexity of the diagnostic algorithm described herein is O=D*(Q+V*SF), wherein O represents the number of outcomes, D represents the number of diagnoses, Q represents the number of questions, V represents the number of vital signs, and SF represents the minimal vital sweep frequency.

This would never actually happen because different vitals and labs are added to the system based on independent timelines. Some items are checked every 5, 10, 15 or 20 minutes and some labs are only performed once, when needed, even though the minimum sweep time can be 5 minutes. This means that there are many fewer data points than could be mathematically available for analysis.

Question and vital pruning can be performed to discard items that would have no effect on a given outcome.

Because the number of outcomes is low and human interactions are slow in the current prototype, an active pruning algorithm was not used to speed up the diagnostic module's analysis and returned sorted diagnoses. There are numerous locations to add time optimizations based on hardware response times and the number of diagnoses that needed to be calculated. These can occur by early pruning of lower probability diagnoses, only recalculating between phase transitions, or leaving lower probabilities diagnoses calculations until after Phase 5b.

### Physical Implementations

Embodiments of the invention can be provided as a stand-alone hardware device or as software for execution on a computing device. Furthermore, embodiments of the invention can be offered over the internet as a service.

Exemplary computing devices include a general purpose computer (e.g., a personal computer (“PC”), laptop, desktop), workstation, mainframe computer system, a patient telemetry device, a smartphone (e.g., a device sold under the IPHONE® trademark by Apple, Inc. of Cupertino, Calif., the WINDOWS® trademark by Microsoft Corporation of Redmond Wash., the ANDROID® trademark by Google Inc. of Mountain View, Calif., and the like), a tablet (e.g., devices sold under the IPAD® trademark from Apple Inc. of Cupertino, Calif. and the KINDLE® trademark from Amazon Technologies, LLC of Reno, Nev. and devices that utilize WINDOWS® operating systems available from Microsoft Corporation of Redmond, Wash. or ANDROID® operating systems available from Google Inc. of Mountain View, Calif.), a video game console (e.g., the WII U® console available from Nintendo of America Inc. of Redmond, Wash.; the SONY® PLAYSTATION® console available from Kabushiki Kaisha Sony Corporation of Tokyo, Japan; the MICROSOFT® XBOX® console available from Microsoft Corporation of Redmond, Wash.), smart speaker devices (e.g., devices sold under the HOMEPOD™ trademark by Apple, Inc. of Cupertino, Calif., the AMAZON ECHO™ trademark from Amazon Technologies, LLC of Reno, Nev., the GOOGLE HOME™ trademark by Google Inc. of Mountain View, Calif., and the CASTLEHUB® trademark by CastleOS Software, LLC of Johnston, Rhode Island), medical devices (e.g., insulin pumps, hospital monitoring systems, intravenous (IV) pumps), electronic medical record (EMR) systems, electronic health record (EHR) systems, and the like.

Such a computing device can include a processor device (or central processing unit “CPU”), a memory device, a storage device, a user interface, a system bus, and/or a communication interface.

A processor can be any type of processing device for carrying out instructions, processing data, and so forth.

A memory device can be any type of memory device including any one or more of random access memory (“RAM”), read-only memory (“ROM”), Flash memory, Electrically Erasable Programmable Read Only Memory (“EEPROM”), and so forth.

A storage device can be any data storage device for reading/writing from/to any removable and/or integrated optical, magnetic, and/or optical-magneto storage medium, and the like (e.g., a hard disk, a compact disc-read-only memory “CD-ROM”, CD-ReWritable “CD-RW”, Digital Versatile Disc-ROM “DVD-ROM”, DVD-RW, and so forth). The storage device can also include a controller/interface for connecting to a system bus. Thus, the memory device and the storage device can be suitable for storing data as well as instructions for programmed processes for execution on a processor.

The user interface can include a touch screen, control panel, keyboard, keypad, display, voice recognition and control unit, or any other type of interface, which can be connected to a system bus through a corresponding input/output device interface/adapter.

The communication interface can be adapted and configured to communicate with any type of external device. The communication interface can further be adapted and configured to communicate with any system or network, such as one or more computing devices on a local area network (“LAN”), wide area network (“WAN”), the internet, and so forth. The communication interface can be connected directly to a system bus or can be connected through a suitable interface.

The computing device can, thus, provide for executing processes, by itself and/or in cooperation with one or more additional devices, that can include algorithms for diagnosing a condition in accordance with the present invention. The computing device can be programmed or instructed to perform these processes according to any communication protocol and/or programming language on any platform. Thus, the processes can be embodied in data as well as instructions stored in a memory device and/or storage device or received at a user interface and/or communication interface for execution on a processor.

The computing device can control the operation of the system components in a variety of ways. For example, computing device can modulate the level of electricity provided to a component. Alternatively, the computing device can transmit instructions and/or parameters a system component for implementation by the system component.

### Implementation in a Tablet or Smartphone

Embodiments of the invention can be implemented on a tablet computer and/or smartphone and can utilize tablet/smartphone components such a digital camera, screen, and the like to implement one or more neurological, ear, nose, & throat (ENT), and dermatological examinations. Exemplary neurological examinations that can be automated using a tablet or a smartphone include: speech evaluation, facial weakness evaluation of upper and lower face, cerebellar/aphasia evaluation, visual acuity evaluation, peripheral vision evaluation, diplopia evaluation, extremity weakness evaluation, and the like. Exemplary ENT examinations include pharyngeal examinations and the like. Exemplary dermatological examinations include jaundice/pallor examination, lesion comparison, and the like.

For example, a computer-implemented neurological examination is advantageously systematic and able to quantify results. In clinical practice, subtle neurological deficits may be overlooked by the patient as well as the physician. For example, a visual field cut may be present without the patient's awareness. Without the patient's complaint to focus the clinician's attention, such a finding could be missed unless a careful and disciplined examination is performed.

### Exemplary Sensors

Embodiments of the invention can interact with one or more sensors that can be integral with or external to a diagnostic system. Exemplary sensors include electrocardiograph (ECG) devices that can measure heart rate and heart rate variability, respiratory rate measurement devices (e.g., based on variation in thoracic impedance), temperature sensors, oxygen saturation sensors, blood pressure sensors, auscultation devices, spirometers, urinalysis devices, mononucleosis test devices, and the like.

Exemplary oxygen saturation and blood pressure measurement devices include conventional pulse oximeters, sphygmomanometers, and the like. In one embodiment, oxygen saturation and/or blood pressure are measured using the device described in U.S. Provisional Patent Application Ser. No. 62/417,231, filed Nov. 3, 2016, and U.S. Provisional Patent Application Ser. No. 62/432,171, filed Dec. 9, 2016. Blood glucose can be measured using the device described in U.S. Provisional Patent Application Ser. No. 62/417,226, filed Nov. 3, 2016, U.S. Provisional Patent Application Ser. No. 62/432,035, filed Dec. 9, 2016, U.S. Provisional Patent Application Ser. No. 62/544,845, filed Aug. 13, 2017, and U.S. Provisional Patent Application Ser. No. 62/573,087, filed Oct. 16, 2017. Hemoglobin can be measured using the device described in U.S. Provisional Patent Application Ser. No. 62/432,037, filed Dec. 9, 2016. White blood cells can be measured using the device described in U.S. Provisional Patent Application Ser. No. 62/432,030, filed Dec. 9, 2016.

### Other Sources of Vital Signs and Objective Values

Vital signs and objective values can also be obtained from conventional methods and standard laboratory tests. Examples of such measurements include urinalysis testing for urine leukocyte esterase, urine nitrites, urine glucose, and urine ketones. Other examples include blood draws (e.g., finger-stick capillary blood draws) for detection of leukocytosis, leukopenia, and the like. Embodiments of the invention can interact with electronic medical records systems, patient or laboratory portals, allow manual input of results, and the like.

### Generation of Reports

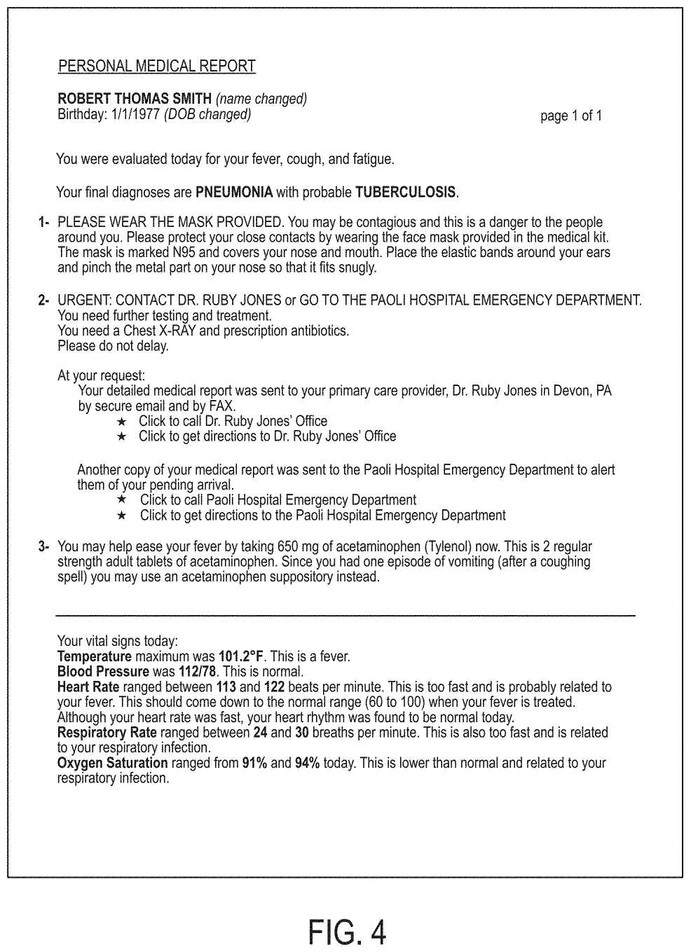

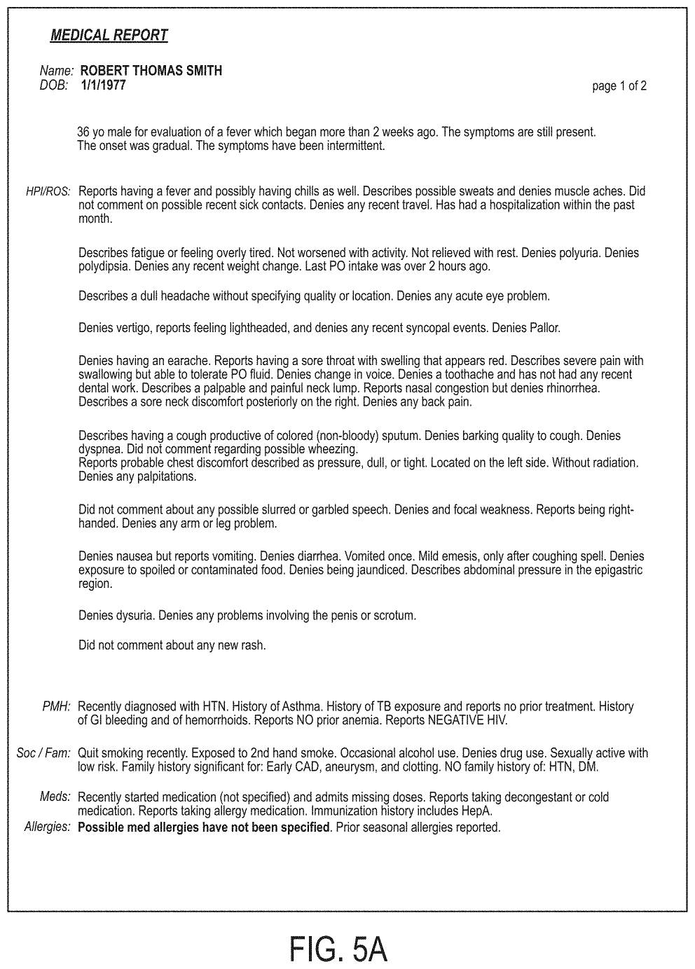

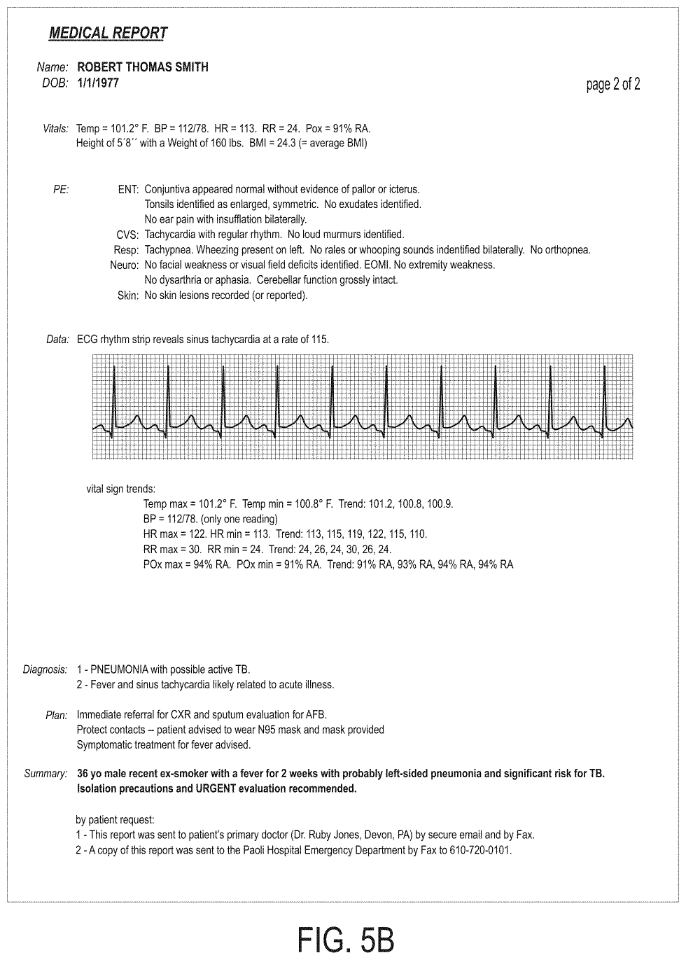

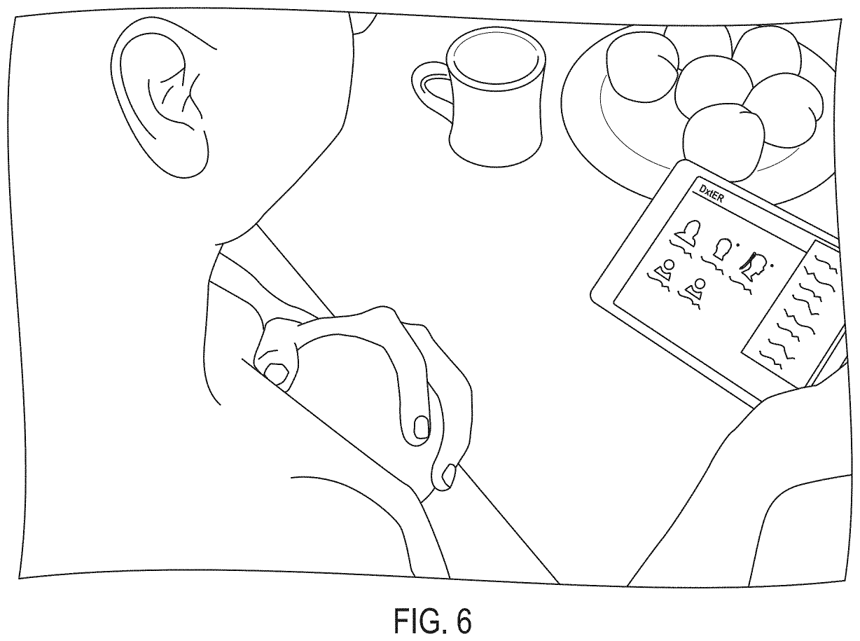

Reports can be generated at the conclusion of a session. The reports can summarize the encounter in a way that easily integrates into a medical portfolio. One exemplary report depicted in FIG. 4 can be written in language for the layman user and include a clear action plan. Another exemplary report depicted in FIGS. 5A and 5B can be a detailed medical report fit to share with the user's primary physician, specialist physician, other medical professional, or as a concise clinical summary when referral is indicated for urgent or emergency care.

Electronic versions can be HL7-compliant and in an encrypted format that can be communicated using various standards and interfaces. FIG. 4 shows the medical report and action plan that would have been generated for a patient diagnosed with suspected tuberculosis. FIGS. 5A and 5B show the detailed medical report for the same case. The name and birthdate displayed in the reports have been changed to protect the individual's privacy.

Along with an action plan, embodiments of the invention can provide the user with condition-specific information with links to connect to detailed and trusted knowledge sources. One exemplary source is UPTODATE®, available from UpToDate, Inc. of Waltham, Mass., a premier evidence-based clinical decision support resource.

Embodiments of the invention can guide users to take urgent action when warranted and provides guidance during emergencies (e.g., choking, CPR, injury, anaphylaxis, allergic reactions, head injuries, concussions, lacerations, orthopedic injuries, burns, heat exposure, heat stroke, cold exposure, frostnip, frostbite, and the like). Embodiments of the invention can help a user find the closest urgent care center or Emergency Department.

Integration with External Systems and Actors

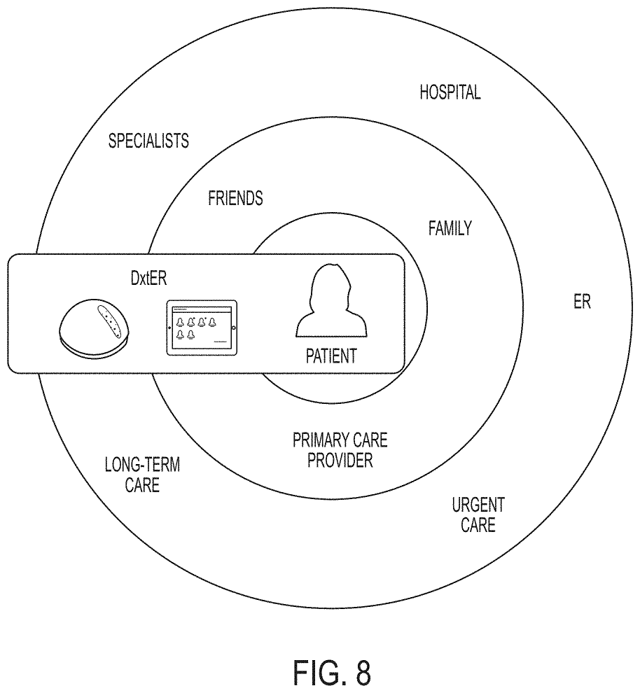

Referring now to FIG. 8, embodiments of the invention (referred to FIG. 8 under the DXTER™ trademark) can help users diagnose and monitor illnesses in the comfort of their own homes and empower them by providing valuable information at the time they need it. Users will be able to make better-informed decisions about their health and will also be able to communicate data seamlessly with their health care providers—e.g., by sending email updates and alerts, and facilitating accurate and up-to-date medical records.

The U.S. health care system is quickly transforming from a system focused on treating illness to one that supports and rewards providers who maintain and improve their patients' health and deliver high quality care at a better value. Growing acceptance of the Patient-Centered Medical Home model, where physicians have responsibility for the comprehensive coordination of care for their patients, rewards clinicians who can improve access and care for their patients. As a more time-intensive model, though, the Patient-Centered Medical Home concept also places additional burdens on physicians.

New generations of physicians understand and accept the requirements of this consumer-centric model because they also embrace the benefits to their patients. Embodiments of the invention will benefit physician practices as they meet these new demands by reducing patient visits for routine or less complex issues. The steady stream of patient-specific data provided by embodiments of the invention will also alert physicians about acute and severe conditions that require immediate treatment and follow-up, and also assist them in ongoing care management for their patients who have chronic ailments.

Emergency Departments and urgent care centers will be less crowded with patients that do not require immediate care. Non-urgent use of Emergency Departments is pervasive and not only clogs system resources, but drives up health care costs. Broad adoption of embodiments of the invention in the home will allow users to obtain the answers they need quickly and efficiently, while avoiding an unnecessary interaction with the health care system. Usage of the invention could reduce ED wait times, result in better deployment of professional staff, lower system costs, and contribute to a more affordable, sustainable model of health care delivery.

Purchasers of healthcare will find that the invention complements value-based purchasing strategies. As the cost of healthcare continues to climb, employers, governments, and others are looking for ways to move from paying for volume to paying for value. Provider reimbursement is rapidly shifting from traditional fee-for-service to paying providers based on performance and patient outcomes. Embodiments of the invention aid providers in maintaining their patients' health remotely and will foster improved, focused communication between patients and their doctors.

By empowering consumers with timely information about their health, embodiments of the invention will also drive improvements in overall population health. In the United States, the single greatest opportunity to reduce preventable deaths and improve population health is to influence individuals' behavior patterns. Healthy choices must be easier for people to make in their daily lives. A growing number of patient-consumers are already gathering data on themselves and sharing daily exercise regimens or food choices with friends and family. New technologies such as the invention will accelerate this trend in consuming personal health data and gamification will be key in persuading people to live healthier lives. Rapid positive behavior change is possible. As new generations of health care consumers become better informed and more involved in their own care, they will become smarter consumers who are better equipped to make healthier lifestyle choices.

Interaction with the User, Sensors, and the Cloud

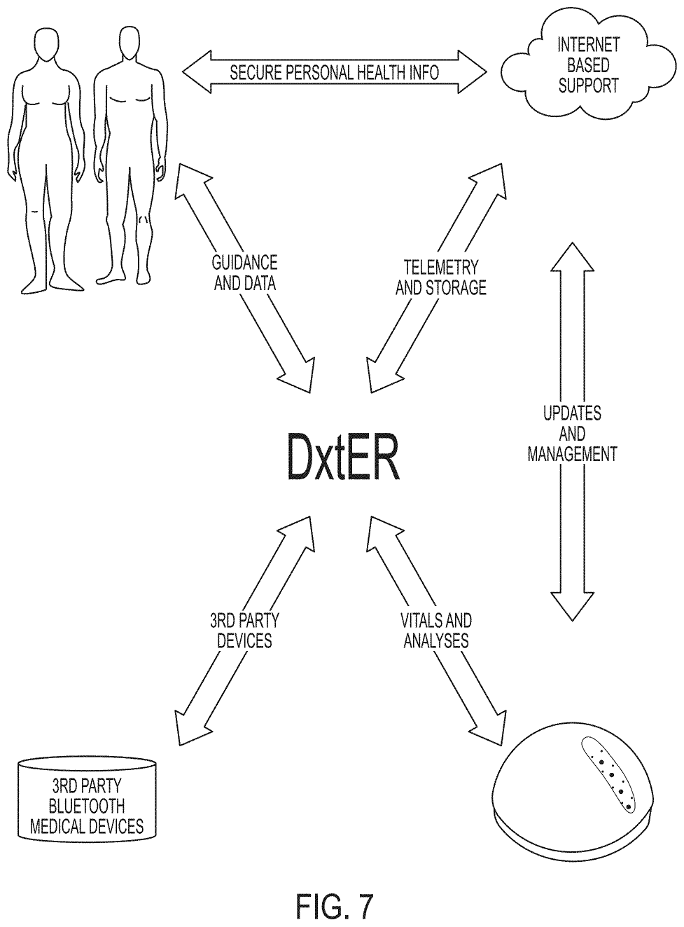

Referring now to FIG. 7, embodiments of the invention can be described as a platform that includes both a series of sensors and software to manage the healthcare needs for its users. Embodiments of the invention can interface with its own sensors and third party devices, e.g., those using various wired or wireless standards such as the BLUETOOTH® standard. Embodiments of the invention can use the internet to provide support and guidance to users, securely store data, and manage software and firmware updates.

### WORKING EXAMPLES

### Working Example 1—Retrospective Matched Case-Control Study

This study quantifies the ability of an artificially intelligent medical diagnostic module to autonomously diagnose 13 conditions. The diagnostic module does not utilize any lab or radiology results to achieve its diagnosis.

### Methods

Test cases and matched control cases were drawn from records of patients evaluated and created in a health system with 4 hospitals and over 170,000 ED visits annually.

### Selection Criteria for Test Cases and Matched Controls

Each test case carried one of 13 specific diagnoses. Each confirmed positive test case was matched to a control case based on age and presenting chief complaint.

The study was reviewed and approved by the health system's Institutional Review Board. Specific selection criteria were strictly followed including the following exclusion criteria.

TABLE 1
Exclusion Criteria

Patients younger than 18 and older than 70 were excluded.
Patients with documented pregnancy were excluded.
Patients that were incarcerated at the time of their visit were excluded.
Patients who were admitted to the Intensive Care Unit or transferred to
another institution were excluded.
Patients who expired in the Emergency Department or during their hospital
stay were excluded.
Patients who were not evaluated and treated between Jan. 1, 2009 and
Dec. 31, 2013 were excluded.

Confirmation of each condition was required. The standards for confirming the presence of a given condition are outlined below.

TABLE 2
Criteria for Inclusion as a Test Case
(These data were not available to the diagnostic module)

| | |
|---|---|
| Pertussis | Positive culture or positive PCR for |
| | Bordetella Pertussis DNA |
| Active Pulmonary | Positive acid-fast bacillus in sputum |
| Tuberculosis | identified as Mycobacterium Tuberculosis |
| Mononucleosis | Positive heterophile antibody test or |
| | Positive Epstein Barr VCA IgM Ab |
| Anemia | Hemoglobin < 8.0 g/dL |
| Urinary Tract Infection | Positive urine culture |
| New or Uncontrolled Diabetes | Glucose > 500 mg/dL |
| Acute Hemorrhagic Stroke | Acute hemorrhage on brain CT in absence |
| | of trauma |
| Acute Otitis Media | Direct visualization documented and |
| | diagnosis by provider |
| Acute Hepatitis A | Positive IgM for Hepatitis A Antibody |
| Pneumonia | Documented verification by radiologist on |
| | chest radiograph or CT scan |
| COPD | Acute flare verified by pulmonary consult |
| Streptococcal Pharyngitis | Positive rapid antigen detection test |
| Atrial Fibrillation | Atrial fibrillation with rapid ventricular |
| | response on 12-lead ECG |

Each test case and its matched control case had similar symptomatology. The matched controls were required to have undergone definitive testing for the given condition. For example, case controls for tuberculosis must have been tested and have a negative culture for acid-fast bacilli.

TABLE 3
Criteria as a Matched Control Case

Each control case must share the same presenting chief complaint with
their matched test case.
Each control case must be within +/−5 years of age of their matched
test case.
Each control case must have documented definitive testing for the
condition of the matched test case.
Each control case must be randomly selected from patients who have
been evaluated and treated in a MAIN LINE ™ Hospital ED between
Jan. 1, 2009 and Dec. 31, 2013.
Each control case must not meet any of the exclusion criteria.

### Artificially Intelligent Medical Diagnostic Module

The artificially intelligent medical diagnostic module synthesized subjective elements from a patient's medical history (e.g., cough, abdominal pain, shortness of breath, etc.) and objective elements from the documented physical examination (e.g., pallor, basic neurological exam elements, etc.) and vital signs (e.g., heart rate, respiratory rate, temperature, pulse oximetry, and blood pressure).

The test system (diagnostic module) did not utilize any lab or radiology results to achieve its diagnosis.

### Results

The results were compared using odds ratios. The odds ratios along with sensitivities, specificities, positive predictive values (PPV), and negative predictive values (NPV) are detailed in Tables 5-13 and summarized in Table 4 below.

For example, 13 test-positive tuberculosis cases were matched with 13 control cases of similar age and symptomatology. For this group of 26 total cases, those with actual active pulmonary tuberculosis were 40 times more likely (or at least 3.6 times with 95% confidence) to have a reported diagnosis or tuberculosis by an embodiment of the invention when compared to their matched control group of similar symptoms.

Table 4 details the performance of an embodiment of the invention for the 13 conditions and highlights the minimum odds ratios.

TABLE 4
Performance of Artificially Intelligent Medical Diagnostic Module

| | | | | | | | Minimum |
|---|---|---|---|---|---|---|---|
| | | | | | | | ODDS |
| | | | | | | | RATIO |
| | | | | | | ODDS | with 95% |
| Condition | N | Sensitivity | Specificity | PPV | NPV | Ratio | Confidence |
| Pertussis | 16 | 87.5% | 75.0% | 77.8% | 85.7% | 21.0 | 1.5 |
| Active | 26 | 92.3% | 79.9% | 80.0% | 90.0% | 40.0 | 3.6 |
| Pulmonary | | | | | | | |
| Tuberculosis | | | | | | | |
| Mononucleosis | 26 | 92.3% | 53.8% | 66.7% | 87.5% | 14.0 | 1.4 |
| Anemia | 24 | 83.3% | 100% | 100% | 85.7% | 105.0 | 4.5 |
| Urinary Tract | 28 | 100% | 71.4% | 77.8% | 100% | 67.7 | 3.3 |
| Infection | | | | | | | |
| New or | 30 | 80.0% | 100% | 100% | 83.3% | 110.7 | 5.2 |
| Uncontrolled | | | | | | | |
| Diabetes | | | | | | | |
| Acute | 18 | 66.7% | 100% | 100% | 75.0% | 35.3 | 1.5 |
| Hemorrhagic | | | | | | | |
| Stroke | | | | | | | |
| Acute Otitis | 22 | 90.9% | 72.7% | 76.9% | 88.9% | 26.7 | 2.3 |
| Media | | | | | | | |
| Acute | 18 | 77.8% | 66.7% | 70.0% | 75.0% | 7.0 | 0.9 |
| Hepatitis A | | | | | | | |
| Pneumonia | 14 | 100% | 71.4% | 77.8% | 100% | 33.0 | 1.3 |
| COPD | 22 | 100% | 100% | 100% | 100% | 529.0 | 9.6 |
| Streptococcal | 34 | 100% | 5.9% | 51.5% | 100% | 3.2 | 0.12 |
| Pharyngitis | | | | | | | |
| Atrial | 30 | 100% | 100% | 100% | 100% | 441.0 | 8.0 |
| Fibrillation | | | | | | | |

TABLE 5
Pertussis (N = 16)

| | Condition | Condition | | |
|---|---|---|---|---|
| | is Present | is Absent | Total | |
| Diagnostic Module | 7 | 2 | 9 | PPV = 77.8% |
| Reports Condition | | | | |
| Present | | | | |
| Diagnostic Module | 1 | 6 | 7 | NPV = 85.7% |
| Reports Condition | | | | |
| Absent | | | | |

| | 8 Test Cases | 8 Matched Controls |
|---|---|---|
| Odds Ratio = 21.0 | Sensitivity = 87.5% | Sensitivity = 75.0% |
| 95% CI = 1.5 to 293.3 | 95% CI = 47.4 to 97.9 | 95% CI = 35.1 to 96.1 |

TABLE 6
Pulmonary Tuberculosis (N = 16)

| | Condition | Condition | | |
|---|---|---|---|---|
| | is Present | is Absent | Total | |
| Diagnostic Engine | 12 | 3 | 15 | PPV = 80.0% |
| Reports Condition | | | | |
| Present | | | | |
| Diagnostic Engine | 1 | 10 | 11 | NPV = 90.9% |
| Reports Condition | | | | |
| Absent | | | | |

| | 13 Test Cases | 13 Matched Controls |
|---|---|---|
| Odds Ratio = 40.0 | Sensitivity = 92.3% | Sensitivity = 76.9% |
| 95% CI = 2.6 to 447.1 | 92.3% CI = 63.9 to 98.7 | 95% CI = 46.2 to 94.7 |

TABLE 7
Mononucleosis (N = 26)

| | Condition | Condition | | |
|---|---|---|---|---|
| | is Present | is Absent | Total | |
| Diagnostic Engine | 12 | 62 | 15 | PPV = 66.7% |
| Reports Condition | | | | |
| Present | | | | |
| Diagnostic Engine | 1 | 7 | 8 | NPV = 87.5% |
| Reports Condition | | | | |
| Absent | | | | |

| | 13 Test Cases | 13 Matched Controls |
|---|---|---|
| Odds Ratio = 14.0 | Sensitivity = 92.3% | Sensitivity = 53.8% |
| 95% CI = 1.4 to 141.5 | 95% CI = 63.9 to 98.7 | 95% CI = 25.2 to 80.7 |

TABLE 8
Anemia (N = 26)

| | Condition | Condition | | |
|---|---|---|---|---|
| | is Present | is Absent | Total | |
| Diagnostic Engine | 10 | 0 | 10 | PPV = 100% |
| Reports Condition | | | | |
| Present | | | | |
| Diagnostic Engine | 2 | 12 | 14 | NPV = 85.7% |
| Reports Condition | | | | |
| Absent | | | | |

| | 12 Test Cases | 12 Matched Controls |
|---|---|---|
| Odds Ratio = 105.0 | Sensitivity = 83.3% | Sensitivity = 100% |
| 95% CI = 4.5 to 2438.8 | 95% CI = 51.6 to 97.4 | 95% CI = 73.4 to 100 |

TABLE 9
Urinary Tract Infection (N = 28)

| | Condition | Condition | | |
|---|---|---|---|---|
| | is Present | is Absent | Total | |
| Diagnostic Engine | 14 | 4 | 18 | PPV = 77.8% |
| Reports Condition | | | | |
| Present | | | | |
| Diagnostic Engine | 0 | 10 | 10 | NPV = 100% |
| Reports Condition | | | | |
| Absent | | | | |

| | 14 Test Cases | 14 Matched Controls |
|---|---|---|
| Odds Ratio = 67.7 | Sensitivity = 100% | Sensitivity = 71.4% |
| 95% CI = 3.3 to 1397.5 | 95% CI = 76.7 to 100 | 95% CI = 41.9 to 91.4 |

TABLE 10
New or Uncontrolled Diabetes (N = 30)

| | Condition | Condition | | |
|---|---|---|---|---|
| | is Present | is Absent | Total | |
| Diagnostic Engine | 12 | 04 | 12 | PPV = 100% |
| Reports Condition | | | | |
| Present | | | | |
| Diagnostic Engine | 3 | 15 | 18 | NPV = 83.3% |
| Reports Condition | | | | |
| Absent | | | | |

| | 15 Test Cases | 15 Matched Controls |
|---|---|---|
| Odds Ratio = 110.7 | Sensitivity = 80% | Sensitivity = 100% |
| 95% CI = 5.2 to 2350.6 | 95% CI = 51.9 to 95.4 | 95% CI = 78.0 to 100 |

TABLE 11
Acute Hemorrhagic Stroke (N = 18)

| | Condition | Condition | | |
|---|---|---|---|---|
| | is Present | is Absent | Total | |
| Diagnostic Engine | 6 | 0 | 6 | PPV = 100% |
| Reports Condition | | | | |
| Present | | | | |
| Diagnostic Engine | 3 | 9 | 12 | NPV = 75.0% |
| Reports Condition | | | | |
| Absent | | | | |

| | 9 Test Cases | 9 Matched Controls |
|---|---|---|
| Odds Ratio = 35.3 | Sensitivity = 66.7% | Sensitivity = 100% |
| 95% CI = 1.5 to 804.5 | 95% CI = 30.1 to 92.1 | 95% CI = 66.2 to 100 |

TABLE 12
Acute Otitis Media (N = 22)

| | Condition | Condition | | |
|---|---|---|---|---|
| | is Present | is Absent | Total | |
| Diagnostic Engine | 10 | 3 | 13 | PPV = 76.9% |
| Reports Condition | | | | |
| Present | | | | |
| Diagnostic Engine | 1 | 8 | 9 | NPV = 88.9% |
| Reports Condition | | | | |
| Absent | | | | |

| | 11 Test Cases | 11 Matched Controls |
|---|---|---|
| Odds Ratio = 26.7 | Sensitivity = 90.9% | Specificity = 72.7% |
| 95% CI = 2.3 to 308.0 | 95% CI = 58.7 to 98.5 | 95% CI = 39.1 to 93.7 |

TABLE 13
Acute Hepatitis A (N = 18)

| | Condition | Condition | | |
|---|---|---|---|---|
| | is Present | is Absent | Total | |
| Diagnostic Engine | 7 | 3 | 10 | PPV = 70% |
| Reports Condition | | | | |
| Present | | | | |
| Diagnostic Engine | 2 | 6 | 8 | NPV = 75.0% |
| Reports Condition | | | | |
| Absent | | | | |

| | 9 Test Cases | 9 Matched Controls |
|---|---|---|
| Odds Ratio = 7.0 | Sensitivity = 77.8% | Specificity = 66.7% |
| 95% CI = 0.9 to 56.9 | 95% CI = 40.1 to 96.5 | 95% CI = 30.1 to 92.1 |

This study assesses the validity of an artificially intelligent medical diagnostic module in its ability to autonomously diagnose a limited set of medical conditions.

The diagnostic module showed the best performance in differentiating between test cases and matched controls for the following 7 conditions (in descending order of reliability): COPD. atrial fibrillation, diabetes, anemia. pulmonary tuberculosis, urinary tract infection, and acute otitis media. The diagnostic module was less able, however still significantly positive, in reliably differentiating between test cases and controls for the following 4 conditions: pertussis, mononucleosis, acute hemorrhagic stroke, and pneumonia.

The diagnostic module evaluated here showed significantly positive results for 11 of the 13 conditions rested. It is believed that with additional objective data, the diagnostic module's ability to reliably diagnose medical conditions could be greatly enhanced.

### Working Example 2—Validation of Artificial Intelligence Logic

To further validate and refine the artificial intelligence logic, Applicant recruited experienced physicians who were given two unique patient personas for each medical condition. This exercise was designed to test if the system would initiate the proper tests and questions and integrate results as expected.

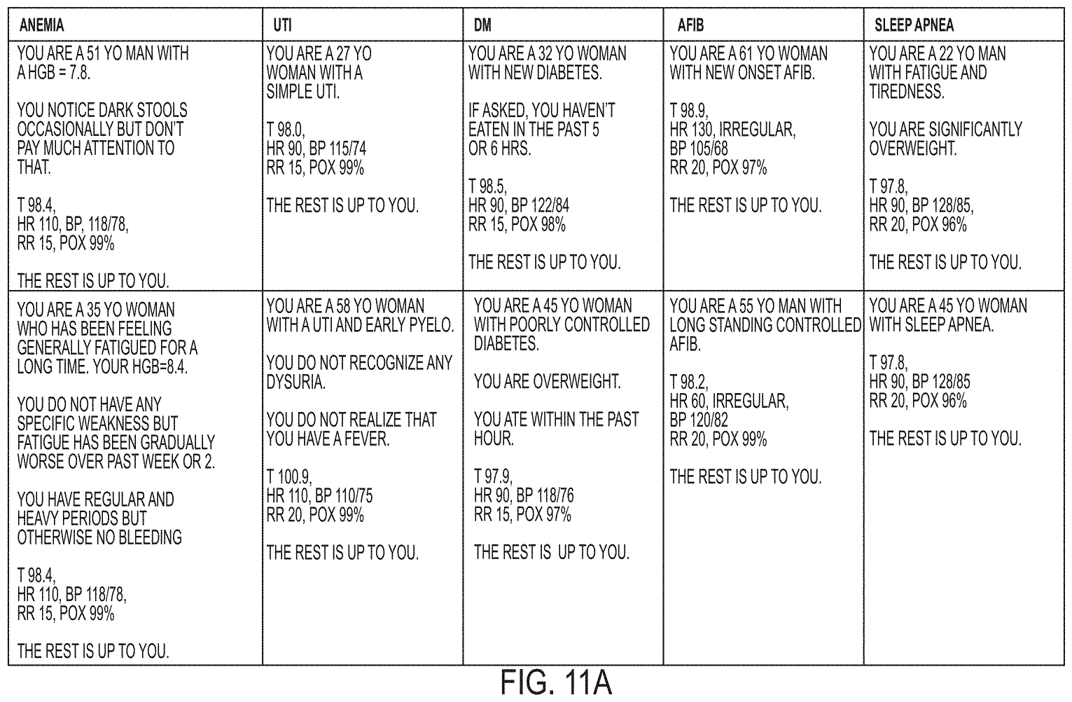

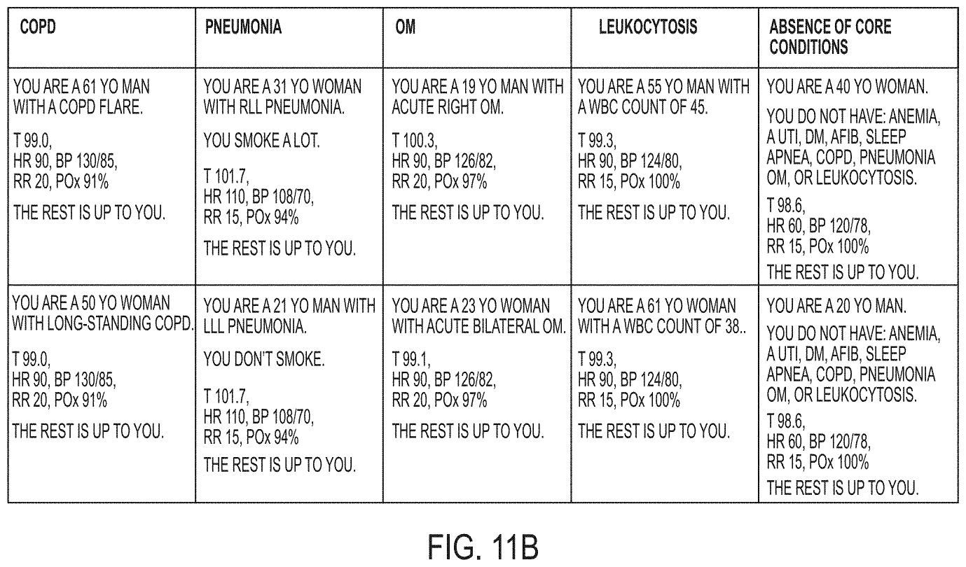

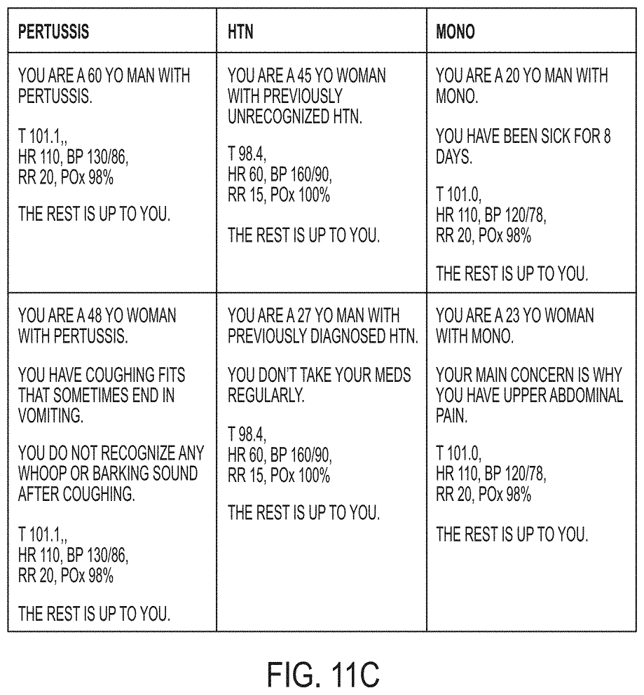

Physicians were unfamiliar with the underlying logic algorithms of the system and were instructed to complete sessions with the software while role-playing each persona. The personas were used as a guide for the expert physician testers. The format allowed the tester to take the session in any direction that they deemed appropriate. The personas are shown in FIGS. 11A-11C.

Live simulated vital signs and any results from any called-upon test were automatically streamed to the expert's test device. These sessions were observed by members of Applicant's team and all results were logged for analysis. Two patient personas were developed for each diagnosis: the first was a classic presentation of the condition and the second was a more difficult or less common presentation of the disease.

The results of the expert sessions with simulated vitals and tests were as follows. For Persona 1 scenarios, the system correctly triggered the required tests and correctly diagnosed 12 of the 13 conditions. A misdiagnosis was counted when the system gave 2 diagnoses for the atrial fibrillation (AFib) case (reporting both AFib and anemia). For Persona 2 scenarios, the system correctly triggered the required tests and again was able to diagnosis 12 of the 13 conditions. The system misdiagnosed pertussis as pneumonia.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

### APPENDIX A—USER PROFILE DATA ELEMENTS

NSNumber *cdFamilyAneurysm;
NSNumber *cdFamilyBloodClotting;
NSNumber *cdFamilyDiabetes;
NSNumber *cdFamilyHeartAttackBefore60;
NSNumber *cdFamilyHeartDiseaseBefore60;
NSNumber *cdFamilyHighBloodPressure;
NSNumber *cdFamilyHypertension;
NSNumber *cdFamilyStrokeBefore60;
NSNumber *cdFamilySuddenDeath;
NSNumber *cdUserActiveSession;
NSNumber *cdUserBMI;
NSDate *cdUserDOB;
NSString *cdUserEmailAddress;
NSString *cdUserFirstName;
NSNumber *cdUserGender;
NSNumber *cdUserHeight;
NSNumber *cdUserKnownAsthmatic;
NSNumber *cdUserKnownCocaineUse;
NSNumber *cdUserKnownCongestiveHeartFailure;
NSNumber *cdUserKnownCOPD;
NSNumber *cdUserKnownDiabetic;
NSNumber *cdUserKnownDrinkingStatus;
NSNumber *cdUserKnownDrugUse;
NSNumber *cdUserKnownHeartAttack;
NSNumber *cdUserKnownHeartDisease;
NSNumber *cdUserKnownHighBP;
NSNumber *cdUserKnownHIV;
NSNumber *cdUserKnownImmunosuppressants;
NSNumber *cdUserKnownKidneyInfection;
NSNumber *cdUserKnownKidneySlowing;
NSNumber *cdUserKnownKidneyStones;
NSNumber *cdUserKnownMarijuanaUse;
NSNumber *cdUserKnownRegularPerscriptions;
NSNumber *cdUserKnownRheumatoidMedication;
NSNumber *cdUserKnownSilicosis;
NSNumber *cdUserKnownTobaccoStatus;
NSString *cdUserLastName;
NSString *cdUserMiddleName;
NSString *cdUserNameSuffix;
NSString *cdUserPassword;
NSDate *cdUserProfileCreation;
NSNumber *cdUserProfileIsHidden;
NSDate *cdUserProfileModifiedDate;
NSNumber *cdUserUniqueID;
NSNumber *cdUserWeight;

### APPENDIX B—SAMPLE QUESTION ELEMENT DATA STRUCTURE

### Before Submission of Answer

```
{
  “questionType” : “<ShowOnce>, <Choice>, <CheckboxSingle>,
<CheckboxMany>, <PhotoTake>, <Phototap>, <PanoramaTake>,
<PanoramaFieldCut>“,
  “questionNumber” : “<4-digit Unique ID>“,
  “questionGroup” : “<Group Description>?optional”,
  “questionText” : “<question text string>”,
  “questionLinks” : [
```

“<4-digit identifier>- ><ANY, NEVER, <4-

digit identifier >>“?optional

],

“questionChoices” : [

```
  {
  “0000” : “Option 0”,
  “0001” : “Option 1”
  }
  ],
```

```
  “questionAnswered” : “ “,
  “questionAudio” : “<audioFileName>?optional”,
  “questionVideo” : “<videoFileName>?optional”,
  “questionImage” : “<staticImageFileName>?optional”,
  “questionSubView” : “<subView Identifier Number>?optional”,
  “questionTimingGroup” : “<4-digit Timing Group
Number>?optional”,
  “questionReportGroup” : “<4-digit Reporting Group
Number>?optional”
  }
```

Modification after Answer

{
...
“questionAnswered” : “answered”
“questionResponse” : “<response string>“
“questionTimeStamp” : “<response time stamp string>“
...
}

### APPENDIX C—SAMPLE VITAL ELEMENT DATA STRUCTURES

### Sample Vital Element Data Structure

```
{
  “vitalNumber” : “<4-digit unique identifier>“,
  “vitalName” : “<internal name string>“,
  “vitalDescription” : “<string to present to user>?optional”,
  “vitalType” : “<integer>, <float>, <choice>“,
  “vitalMin” : “<Minimum Value>?optional”,
  “vitalMax” : “<Maximum Value>?optional”,
  “vitalGroup” : “<2-digit group ID>?optional”,
  “vitalChoices” : [
```

{
“0000” : “No Value”,
“0001” : “Option 1”,
“0002” : “Option 2”,
“0003” : “Option 3”,
“0004” : “Option 4”
} ? optional
],

```
  “vitalBoundaryType” :
“<averageValue>, <minimumValue>, <maximumValue>?optional”,
  “vitalDescriptionText” : “<user presented text description of
vital>?optional”,
  “vitalDescriptionBoldItems” : [
```

“<items to bold in

description>?optional”

]

}

### Sample Vital Data Recording Element

```
“vitalRecording” : [
    {
      “vitalValue” : “<string representation of numerical
data>”,
      “recordingTimeStamp” : “<string representation of
entry timestamp>”,
      “instrumentName” : “<instrument Name>?optional”,
      “instrumentSerialNumber” : “<instrument serial
number>?optional”,
      “instrumentVersion” : “<instrument version
number>?optional”
    }
  ],
```

### APPENDIX D—SAMPLE DIAGNOSTICS DATA STRUCTURE

### Sample Diagnosis Data Structure

```
  {
    “diagnosesRunningScore”: “0”,
    “diagnosesName”: “<simplified medical term>“,
    “diagnosesID” : “<unique medical ID number>“,
    “conditionID” : “<XPrize condition identifier>“,
    “diagnosesShortHand” : “<medical shorthand ID string>“,
    “diagnosesQuestionMatrix” : [
      “<4-digit question ID Number>“ “=“ “<4-digit selection
value, GREATERTHAN<float value>, LESSTHAN<float value>, YES>“
“:” “<diagnosis change - float value>“,
examples:
    “0000=0024:.5”,
    “0001=Yes:0”,
    “0013=GREATERTHAN+18.5:1.5”,
],
  “diagnosesVitalMatrix”: [
    “<4-digit vital ID number> <“=“, “<“, “>“> <float value>
“:” <diagnosis change - float value>
examples:
    “0021=0:2”,
    “0022>4:1”,
    “0023<3:3”
],
  “diagnosesGuidancePrimary”: {
    “guidancePart1” : “<Primary Guidance Part 1>“,
    “guidancePart2” : “<Primary Guidance Part 2>“
    “guidancePart3” : “<Primary Guidance Part 3>“
    },
  “diagnosesGuidanceSecondary”: {
    “guidancePart1” : “<Secondary Guidance Part 1>“,
    “guidancePart2” : “<Secondary Guidance Part 2>“
    “guidancePart3” : “<Secondary Guidance Part 3>“
    },
  “diagnosesGuidanceConcerns” : {
    “guidancePart1” : “<Concerns Guidance Part 1>“,
    “guidancePart2” : “<Concerns Guidance Part 2>“
    “guidancePart3” : “<Concerns Guidance Part 3>“
    },
  “diagnosisInformationStartText” : “<diagnosis lead text
string>
  “diagnosisInformationTopGuideText” : “<diagnosis guidance
text string>
  “diagnosisAdditionalInformation” : “<medical shorthand info
string>“,
  “diagnosisBasicInformation” : “<basic medical info string>“
  },
```

1. A computer-implemented method for diagnosing a condition on a computer including a processor, memory, at least one of a user interface and a communication interface, a user profile module, a question module, a vitals module, and a diagnostic module, the computer-implemented method comprising:
(a) controlling the processor and one or more of the user interface, the communication interface, the user profile module, the question module, and the vitals module on the computer to receive one or more inputs regarding a subject's symptoms;
(b) controlling the processor and the diagnostic module on the computer to update a plurality of models stored in the memory on the computer in parallel based on the one or more inputs, each model generating a numerical score reflecting a likelihood of one of a plurality of conditions;
(c) controlling the processor and the diagnostic module on the computer to identify one or more most-likely conditions as a function of the numerical scores produced by the models stored in the memory on the computer;
(d) controlling the processor, the question module, and at least of the user interface and the communication interface on the computer to request additional input based on the most-likely conditions;
(e) controlling the processor, the question module, and at least of the user interface and the communication interface on the computer to receive the additional input;
(f) controlling the processor and the diagnostic module on the computer to update the models stored in the memory on the computer in parallel based on the additional input;
(g) controlling the processor and the diagnostic module on the computer to compare updated numerical scores or a difference between sequenced updated numerical scores to a stored confidence threshold; and
(h) controlling the processor and the diagnostic module and the question module on the computer to repeat steps (c)-(g) until the compared numerical scores or the difference between sequenced numerical scores exceeds the stored confidence threshold.

2. The computer-implemented method of claim 1, wherein the models are weighted summations of the inputs.

3. The computer-implemented method of claim 2, wherein the weighted summations include negative, zero, and positive weightings.

4. The computer-implemented method of claim 1, wherein the models produce a numerical score.

5. The computer-implemented method of claim 4, wherein the confidence threshold is a predefined number that the numerical score must equal or exceed.

6. The computer-implemented method of claim 4, wherein the confidence threshold is a predefined difference between the numerical scores of a most-likely condition and a next-most-likely condition.

7. The computer-implemented method of claim 1, further comprising:
(h) receiving one or more external inputs; and
(i) updating the models based on the one or more external inputs.

8. The computer-implemented method of claim 7, wherein the one or more external inputs include one or more selected from the group consisting of: epidemiological data, subject-entered data, and electronically obtained data.

9. The computer-implemented method of claim 8, wherein the electronically obtained data is periodically updated.

10. A computer-implemented method for diagnosing a condition on a computer including a processor, memory, at least one of a user interface and a communication interface, a user profile module, a question module, a vitals module, and a diagnostic module, the computer-implemented method comprising:
(a) controlling the processor and one or more of the user interface, the communication interface, the user profile module, the question module, and the vitals module on the computer to obtain one or more subject inputs regarding a subject;
(b) controlling the processor and one or more of the user interface, the communication interface, the user profile module, the question module, and the vitals module on the computer to obtain one or more family inputs regarding the subject's family;
(c) controlling the processor and one or more of the user interface, the communication interface, the user profile module, the question module, and the vitals module on the computer to obtain one or more symptom inputs regarding the subject's symptoms;
(d) controlling the processor and the diagnostic module on the computer to update models stored in the memory on the computer for a plurality of conditions based on the one or more subject inputs, family inputs, and symptom inputs;
(e) controlling the processor and the diagnostic module on the computer to identify m most-likely diagnoses based on the models stored in the memory on the computer;
(f) controlling the processor and at least one of the diagnostic module and the question module on the computer to identify n most-influential questions for the m most-likely diagnoses based on previously assigned weights associated between the m most-likely diagnoses and a plurality of questions;
(g) controlling the processor, the question module, and at least of the user interface and the communication interface to present the n most-influential questions to the subject;
(h) controlling the processor, the question module, and at least of the user interface and the communication interface on the computer to obtain responses to at least one of the n most-influential questions;
(i) controlling the processor and the diagnostic module on the computer to update the models stored in the memory on the computer based on the obtained responses;
(j) controlling the processor and the diagnostic module on the computer to update the m most-likely diagnoses based on the models stored in the memory on the computer;
(k) controlling the processor and the diagnostic module on the computer to determine whether a most-likely diagnosis exceeds a confidence threshold and:
(1) if so, controlling the processor and at least of the user interface and the communication interface on the computer to present the most-likely diagnosis to the subject; and
(2) if not, controlling the processor and the diagnostic module and the question module on the computer to repeat steps (f)-(k).

11. A system for diagnosing a condition, the system comprising:
a user profile module comprising computer-readable program code including steps for:
obtaining one or more user profile inputs regarding a subject's medical status and history; and
recording the one or more user profile inputs;
a vitals module comprising computer-readable program code including steps for:
receiving one or more vitals inputs from one or more sources selected from the group consisting of: sensors and laboratories;
determining whether the one or more vitals inputs are clinically significant; and
if the one or more inputs are clinically significant, recording the one or more vitals inputs;
a diagnostic module comprising computer-readable program code including steps for:
populating and updating a plurality of diagnosis modules based on the one or more user profile inputs stored by the user profile module and the one or more vitals inputs stored by the vitals module;
identifying m most-likely diagnoses based on the diagnosis models;
updating the plurality of diagnosis models based on responses to questions posed to the subject by the question module and any further vitals inputs;
updating the m most-likely diagnoses based on the models;
determining whether a most-likely diagnosis exceeds a confidence threshold and:
if so, presenting the most-likely diagnosis to the subject; and
if not, instructing the question module to ask further questions based on the updated m most-likely diagnoses; and
a question module comprising computer-readable program code including steps for:
identifying n most-influential questions for the m most-likely diagnoses based on previously assigned weights associated between the m most-likely diagnoses and a plurality of questions;
controlling a user interface to present the n most-influential questions to the subject;
obtaining responses to at least one of the n most-influential questions; and
recording the responses.
